(19) United States
(12) Patent Application Publication
(10) Pub. No.: US 2007/0280998 A1
(43) Pub. Date: Dec. 6, 2007

(54) Dairy product

(76) Inventors: Larry Milligan, Guelph (CA); John Osborne, Elmira (CA); Moni Eino, Mississauga (CA)

Correspondence Address: GOWLING, LAFLEUR HENDERSON LLP, ONE MAIN STREET WEST, HAMILTON, ON, L8P 4Z5, US

(21) Appl. No.: 11/445,186

(22) Filed: Jun. 2, 2006

(51) Int. Cl. A61K 35/20; A23C 9/12; A23C 9/154

(52) U.S. Cl. 424/439; 424/535; 426/34; 426/580

(57) ABSTRACT

The present invention provides a novel feedstock and method of feeding dairy cows. Milk products derived from cows fed the feedstock are also encompassed. Milk comprising at least 0.3% DHA/g of fatty acids and food products containing components of that milk are also included. The milk product of the invention also contains elevated levels of DPA and EPA.

### FIELD OF INVENTION

The present invention relates to dairy products and food products derived from those dairy products that contain elevated levels of DHA, EPA and DPA. The invention also related to a composition and method for feeding dairy cattle to achieve enhanced PUFA levels in milk.

### BACKGROUND OF THE INVENTION

Omega-3 fatty acids (n-3 FA) are important nutrients necessary for maintaining human health from conception to childhood and throughout life. Docosahexaenoic acid (DHA; C-22:6 n-3), an important n-3 FA, is a major component of nerve, brain and eye tissue. DHA has been associated with a variety of human health benefits including the prevention and management of cardiovascular disease in adults, improved visual acuity in infants, management of inflammatory disease in adults and help with attention deficit disorder. Other omega-3 fatty acids that have been shown to have beneficial physiological effects when consumed, include eicosapentanoic acid (EPA) and docosapentanoic acid (DPA).

The diets of people in many countries and cultures have been shown to be deficient in omega-3 fatty acids. The American Heart Association recommends the daily consumption of fish or fish oil supplement for adults to achieve a combined DHA and EPA (EPA; C20:5 n-3) intake of about 900 mg/d. The current mean intake of DHA and EPA (combined) for adults in North America (which includes about one fish serving every 10 days) is approximately 130 mg/d or 14% of the target intake.

Omega-3 fatty acids have been shown to be required nutrients for optimal maturation of visual and cortical function in human infants. Evidence suggests that breast fed infants have a long-term advantage in cognitive development over formula fed infants. There is some suggestion that the deficiency of long chain polyunsaturated fatty acids such as DHA in infant formulas and cow's milk may be an important factor correlating with these observations.

It is therefore apparent that it would be desirable to add omega-3 acids and/or their sources to ingestable formulations. However, several characteristics make their inclusion in good tasting food a challenge. Omega-3 fatty acids such as DHA, EPA, and DPA are typically derived from a fatty fish and are often used in the form of fish oil with the attendant flavour and odour being major barriers to use. Additionally, these fatty acids containing multiple conjugated double bonds are extremely susceptible to oxidation and rancidity. Thus, their purification, concentration and storage is very difficult As a result, a DHA-containing fish oil, or in vitro cultured algae, which is subjected to concentration, purification, and anti-oxidation is very costly to produce and this also limits their use.

Various types of nutritional supplements containing DHA have been developed. These are usually provided in a compressed tablet or capsule format. While these supplements do provide health benefits, they are still associated with a fishy flavor. An increasing area of interest is food products such as fat spreads like margarine, instant powder concentrates, liquid egg preparations and bread containing DHA. It has been found however that while food products with strong flavors and/or sweetness can significantly mask the unpleasant fishy taste, milder tasting products are unsuitable for this type of enrichment due to poor palatability. In addition, encapsulated oil tends to separate, particularly from liquid products.

Bovine milk and dairy products play an important role in human health and nutrition. The Canada Food Guide recommends the daily consumption of dairy products for people of all ages. Consumption of milk is particularly important for children. In fact, milk is by far and away the primary source of fatty acids for children. Thus, it would be desirable to provide omega-3 enhanced dairy products for consumption from conception throughout life. However, addition of exogenous fish oil to milk results in a foul taste.

Several attempts have been made to provide milk-like products or other products that do contain omega-3 fatty acids.

U.S. Pat. No. 6,727,373 discloses a microbial polyunsaturated fatty acid (PUFA)-containing oil with a high triglyceride content and a high oxidative stability. In addition, a method is described for the recovery of such oil from a microbial biomass derived from a pasteurized fermentation broth, wherein the microbial biomass is subjected to extrusion to form granular particles, dried and the oil then extracted from the dried granules using an appropriate solvent.

U.S. Pat. No. 6,428,832 relates to a process for the preparation of a PUFA-containing food, such as an infant formula, where a composition comprising a PUFA is added at a late stage of the infant formula preparation process. In this way, the PUFA's are minimally exposed to conditions during the process that induce degradation of the PUFA's.

United States Patent Application No. 2004/0131727 provides dairy products containing fish-oil originated EPA and/or DHA and having oxidation and emulsification stability. The soy is acidified by addition of an acid, fermented milk, or acidified milk containing any of the milk acidified by addition of an acid and the fermented milk. The acidified milk contains EPA and/or DHA as fish oil, preferably purified fish oil or fish oil containing EPA and/or DHA in adjusted amount. The acidified milk is produced through an emulsification process. Preferably, the emulsification is performed after a fermentation process of the acidified milk process through a two-stage emulsification process. A food product containing the acidified milk of the invention is also provided.

U.S. Pat. No. 5,976,606 provides a process for producing a DHA (docosahexaenoic acid)-containing tofu or soybean milk drink, or a dry powder thereof that is stable and inexpensive. A DHA-containing fish oil emulsion is prepared by mixing soybean milk with a DHA-containing fish oil at a weight ratio of 1:0.2 to 1:1, and stirring the mixture. The soybean milk drink can be produced by further diluting this emulsion with soybean milk to give a predetermined DHA concentration. DHA-containing tofu can be produced by coagulating the soybean milk containing this emulsion with the addition of a coagulant. Since the same soybean milk as the starting material of tofu or soybean milk drink is used as an emulsifying agent, even if DHA is contained at a high concentration, this does not influence the taste or properties of the product. A dry powder can be obtained by freeze-drying, vacuum drying or spray-drying this tofu or soybean milk drink as required.

United States Patent Application No. 2004/0265462 provides an infant milk formula having long chain polyunsaturated fatty acids, sialic acids, and cholesterol.

U.S. Pat. No. 6,596,302 provides methods for providing nutrition and for enhancing neurological development of preterm infants. Also disclosed is an improved nutritional composition containing specified amounts of DHA and M as well as their precursor (in some species) fatty acids, alpha-linolenic and linoleic acids. The method involves feeding LCP supplemented, nutrient-enriched formulas for an extended feeding regimen, typically until at least 3 months corrected age (CA), preferably to 6 or even 12 months CA. The neurological developments, for example, visual development, motor development and language development were enhanced without findings of anthropometric growth faltering or inhibition.

### SUMMARY OF THE INVENTION

The present invention addresses the problems of the prior art by providing a novel milk product that contains elevated levels of polyunsaturated FA (PUFA), particularly DHA, EPA, and/or DPA. The invention also provides a feed additive to be fed to cattle to achieve this type of milk as well as methods of promoting health and preventing or treating diseases by ingestion of the DHA/EPA/DPA enriched milk.

In one aspect the invention provides a milk product comprising at least 0.3% DHA/g of total milk fatty acids (FA). In a preferred embodiment, the milk comprises at least 0.35%, more preferably 0.4% DHA.

In another aspect, a product comprising at least 0.1% EPA/g of total milk FA is provided.

In a further aspect, a product comprising elevated (at least 0.1%) DPA as a percent of weight of total milk FA is provided.

In a preferred aspect, a milk product comprising at least 0.3% DHA and at least 0.1% EPA/g total milk FA is provided.

In a preferred embodiment, dairy product comprises a product selected from the group consisting of milk, yogurt, cheese, cream, spread, butter, frozen dessert, and baby formula.

A milk product comprising therapeutic levels of DHA & EPA & DPA is also provided.

In an aspect of the invention, a method of obtaining a dairy product comprises feeding to a dairy cow a composition comprising fishmeal having at least 1 g DHA100 g FA, preferably 1.2 g DHA/100 g FA.

A method of preventing or treating cardiovascular disease in humans comprising administering a milk product having at least 0.3% DHA as a percent of total milk FA is also provided.

A feed product for dairy cows comprising a source of PUFA and a protectant is also provided.

A feed product is provided wherein the fishmeal comprises at least 1 g. DHA/100 g FA, preferably at least 1.2 g DHA.

The feed product of the invention further includes a protectant selected from the group consisting of chitin, chitosan, feathermeal, zein, monensin, lignin, metal salts and combinations thereof.

In another aspect of the invention, a food product comprising DHA and/or EPA and/or DPA enriched dairy product is provided.

The food product is preferably selected from the group consisting of soup, sauce, gravy, prepared vegetables, and frozen meals.

### BRIEF DESCRIPTION OF THE DRAWINGS

These and other features of the invention will become more apparent from the following description in which reference is made to the appended drawings wherein:

FIG. 1 illustrates graphically the effect of various protectants on PUFA levels in milk.

### DETAILED DESCRIPTION

Milk is an excellent vehicle by which to achieve widespread human intake of essential nutrients. Not only is milk consumed directly by all age groups, but is widely used in the preparation of other dishes. Preliminary work done at the University of Guelph demonstrated that when DHA is provided to cows in their diet, the DHA is incorporated into the milk and the problem of an unpleasant flavour of the milk is completely eliminated. As described in U.S. Pat. No. 5,932,257, when dairy cattle are fed a feed additive comprising a source of DHA and an inhibitor of microbial degradation of DHA in the rumen comprising feathermeal, the expressed milk from the dairy cattle is enriched for DHA. While this method provides a source of milk that contains DHA, there remained a need for improved methods of getting higher levels of DHA and other healthy polyunsaturated fatty acids into milk.

Milk fat is synthesized either from FA which are taken up from the blood (60%) or by de novo synthesis in the mammary gland (40%). PUFA are not synthesized by ruminant tissues, so their concentration in milk is dependent on how much they ingest and on the amount that flows out of the rumen. The rumen, however, is a site of intense microbial lipid metabolism. Under normal conditions, PUFA like DHA, are hydrogenated by rumen bacteria via a process called biohydrogenation. This results in a very low level of unsaturated FA reaching the small intestine where they can be absorbed.

During the various steps of rumen biohydrogenation (hydrolysis, isomerization and hydrogenation), numerous intermediate compounds are produced. Research has shown that some isomers cause serious milk fat depressions even when small amounts (10 grams or less) reach the small intestine. The presence of certain FA in the mammary gland is thought to interfere with enzymes (Acetyl CoA carboxylase, fatty acid synthase, stearolyl CoA desaturase) involved in de novo fatty acid synthesis. Thus, the problem of getting milk enriched for PUFA without decreasing the total milk fat levels is not easily solved.

The presence of PUFA in milk is affected by two main factors: levels of PUFA in the diet and the presence of other components in the diet that help the PUFA avoid rumen biohydrogenation.

The present invention addresses each of these factors to synergistically obtain an improved product.

The first category includes factors that affect the amount of dietary PUFA offered to dairy cows. This involves the formulation of ruminant diets using PUFA-containing ingredients in sufficient quantities to achieve the desired level in milk. The second category includes any factors that affect the protection of dietary PUFA from rumen biohydrogenation, allowing greater transfer of intact PUFA to the small intestine and ultimately the mammary gland for inclusion in milk.

A preferred source of FA for use in a feedstock of the invention is fishmeal. The form in which FA are present in the fishmeal is key. Lipid fraction analysis of fishmeals revealed that the form of omega-3 FA may vary significantly in different sources. An analysis of these samples is shown in Tables 1A, 1B and 1C. In fishmeal the omega-3 FA are present in a variety of forms: free fatty acids (FFA), triglycerides (TG) and phospholipids (PL). In the feedstock of the present invention, the fishmeal comprises at least 7% FA. The majority of the DHA is preferably associated with the phospholipid fraction. Preferred sources of FA comprise at least 1 g DHA per 100 g FA. More preferred sources comprise at least 1.2 g DHA/100 g FA. Fishmeal is one preferred source of DHA and other FA, however other sources such as fish oil and algae may also be used.

The second component that affects the levels of PUFA transferred from the diet to milk is a protectant. As used herein, the term “protectant” refers to any agent that protects PUFA in the digestive tract and enhances uptake so that the PUFA is transferred to an animal food product for human consumption.

In one embodiment, the protectant takes the form of a metal salt, such as a calcium or magnesium salt, of a PUFA source such as fish oil.

In another aspect of the invention the protectant is an ionophore. Ionophores have been shown to effect rumen fermentation and are sometimes used for improving feed efficiency of milk protein production, reducing milk fat percentage and minimizing loss of body condition in dairy cattle. The present invention provides, for the first time, the use of an ionophore in a feed to enhance PUFA levels in milk. A preferred ionophore for use in the present invention is monensin. Monensin is a cyclic polyether antibiotic derived from Streptomycin cinnamonensis. The feed typically comprises from about 8 to 24 mg monensin per kg of complete diet. A preferred ionophore for use in the invention is Rumensin™

In another aspect of the invention, the protectant is feathermeal. In a preferred embodiment, the ratio of fishmeal to feathermeal is about 5:0.5, preferably about 4:1. Defatted feathermeal comprising less than 7% fat is preferred. Feathermeal comprising less than 5% fat is more preferred. Previous work has shown that feathermeal can act as a good rumen-bypass agent. Animals were fed fishmeal at 1.2 kg/animal/day as a DHA source and feathermeal at 0.3 kg/animal/day as a DHA protectant. The present invention demonstrates that, by using an appropriate source of PUFA, levels of fishmeal can be lowered to about 1.1 to 0.9 kg/animal/day and feathermeal can be lowered about to 0.275 to 0.225 kg/animal/day and still achieve milk levels greater than 0.3% DHA/g of total milk FA without reducing total milk fat levels. This provides a significant commercial value since increased benefits can be achieved at a lower cost.

In yet another aspect, fishmeal itself may act as a protectant for the PUFA it contains. This enables one to significantly reduce the levels of other protectants to be added.

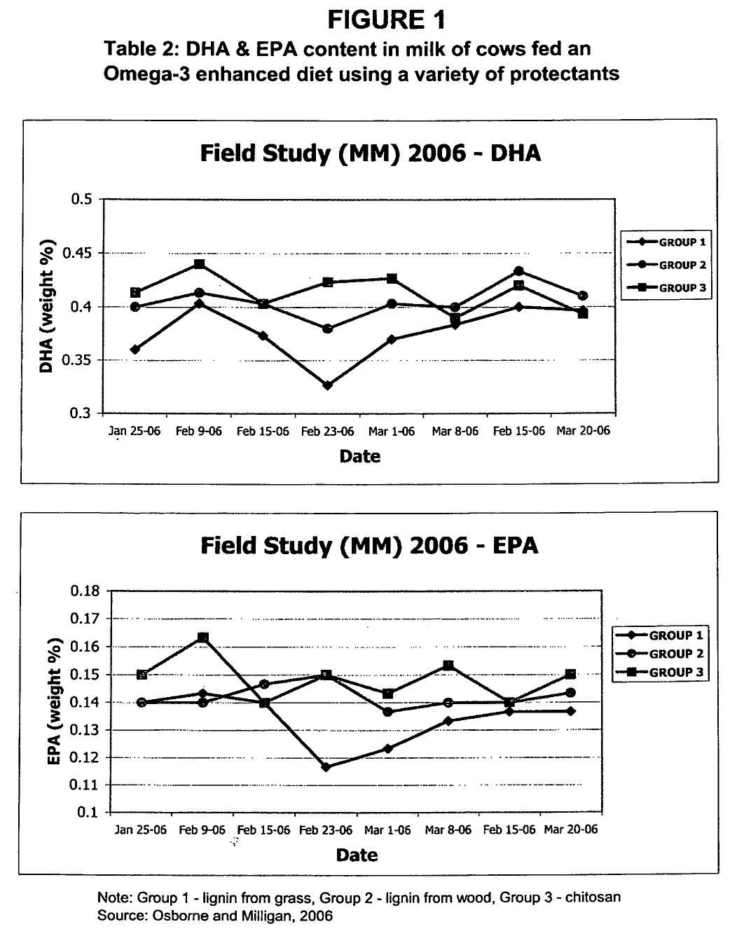

In a further aspect of the invention, the protectant is chitin or chitosan. The chitin or chitosan is typically used in the feed in an amount ranging from about 0.25% to 2.0% on a dry weight basis. The results of an exemplary study are shown in FIG. 1. These results demonstrate that chitosan can act as an effective protectant and that its use as a protectant leads to significantly elevated levels of DHA, EPA and DPA.

In yet another aspect of the invention, lignin is used as a protectant to achieve elevated levels of PUFA in milk. As used herein, the term “lignin” has its normal connotation. It refers to natural lignin and lignin recovered from alkali pulping black liquors such as kraft lignin, soda lignin and the like. The term also encompasses modified lignins. Various polymer sizes of lignin, as well as various molecular forms can be used. The lignin is typically used in the feed in an amount ranging from about 0.25% to 2.0%, preferably about 0.5 to 1% on a dry weight basis. The results of a field trial where lignin was used as a protectant are also shown in FIG. 1. The results demonstrate that lignins from various sources are effective protectant agents that allow dietary PUFA to be transferred to milk.

The protectants for use in the invention may be used singly or in combination. For example, it may be desirable to combine a PUFA metal salt with monensin. Alternatively Rumensin™ may be included in a feedstock comprising feathermeal as a protectant. Combinations of protectants may act additively or synergistically.

The invention also provides a method of producing milk having elevated levels of PUFA, particularly DHA, DPA, and EPA. The method comprises feeding a dairy cow a dietary source of PUFA. The PUFA may be derived from various sources. In a preferred embodiment the PUFA source is fishmeal comprising at least 7% FA. DHA is used as a reference point to predict total PUFA levels. Preferred fishmeal for use in the invention comprises at least 1.2 grams DHA per 100 grams FA. Animals are fed between about 0.8 and 1.2 kilograms of the fishmeal per day. By using the preferred source of fishmeal the total amount can be reduced below standard levels and high levels of PUFA in the milk can still be attained without a reduction in the total milk fat. The PUFA is combined with a protectant such as a metal salt, feathermeal, zein, chitin, chitosan, lignin, fishmeal, an ionophore or mixtures thereof. Animals are fed a nutritionally balanced ration which includes a feedstock or supplement comprising; i) about 0.5 to 10% PUFA prefers 1-8, more preferably 2-5%; and ii) a protectant selected from the group consisting of feathermeal, chitin, zein, chitosan, lignin and monensin.

The feed supplement of the invention may be used either as a top dressing or as part of a total mixed ration. Good results for cows were found by feeding a total of about 1 to 5 kg preferably about 1.5 to 3.5 kg, more preferably about 2 kg, of a feed supplement per day. Additional components such as proteins, vitamins, minerals, buffers, ionophores and combinations thereof may be included in the feed supplement. The feed supplement of the present invention may vary in any one or all of the components described above. Further, one or more of the components may be absent and other components may be present in the feed supplement. In an embodiment of the present invention, the analysis for any one or all of the components may vary by about 20%, preferably less than 10%, more preferably less than 5% of the totals listed above. Amounts may be adjusted for other ruminants such as goats or sheep.

The cows are typically fed the supplement for about 4 to 12 weeks (during the transition from a conventional feeding program to an Omega-3 feeding program) to achieve levels of DHA in excess of 0.3%/g total milk fat. The timing may vary from herd to herd. There may be an initial drop in milk fat content followed by a recovery. Particularly good results have been achieved by 10 weeks.

The invention also encompasses animal products such as milk, meat and processed products e.g. cheese, butter produced by the method of the present invention that exhibit enhanced levels of PUFA including DHA, EPA and DPA. The present invention provides, for the first time a milk containing elevated levels of DHA plus EPA plus DPA. The milk of the invention comprises at least 0.3% DHA, preferably at least 0.35% DHA, more preferably at least 0.44% as a percent of total milk FA. A preferred milk also includes at least 0.1% EPA and 0.1% DPA. Although DHA and other PUFAs are notoriously subject to oxidative degradation, the levels are very stable when secreted in milk. Milk, according to the invention, withstands ultra high temperature pasteurization and also has a subsequent shelf life in excess of 21 days. While the first obvious commercial product is fluid milk, other products are also encompassed. The milk, milk solids, cream and milk fat enriched in PUFA can also be ingredients for other food products. The present invention demonstrates for the first time that PUFA levels achieved in milk products by the methods of the invention remain stable for extended periods of time (i.e. greater than months). The levels remain stable even when the milk product is further processed such as by high temperature, for example, pasteurization or natural cooking. The milk product can be incorporated into processed foods such as cream, butter, ice cream, cheese, yogurt, soups, sauces, spreads, etc. Prepared foods such as packaged, potatoes or pasta and sauce products are also encompassed. This includes dried products. PUFA-enhanced milk products of the invention are particularly useful for inclusion in infant nutritional formulations.

In addition to enhancing the levels of PUFA in animal-derived products, the feed supplement of the invention provides health benefits to livestock as compared to standard feeds.

In addition, the food products derived from animals fed the feed may provide health benefits to humans.

While the methods and compositions of the invention have been described with particular reference to dairy cattle and the production of dairy products, it is clearly apparent that the methods and compositions can also be applied to other ruminants such as beef cattle, sheep and goats.

Dairy farmers are compensated based on the percent of fat in milk. A disadvantage of previous feeds containing PUFA is that they result in a decrease in the percent of total fat (from 3.8 to 2.8%) in milk, with no recovery. The synergistic compositions and methods of the present invention demonstrate that after an initial milk fat depression in some cases, the original milk fat level can be completely recovered in time (usually within 10 weeks). The present invention provides, for the first time, a feedstock and a method of feeding dairy cows whereby omega-3 fatty acids are fed to the animal and transferred to the milk without decreasing the total amount of fat in the resultant milk.

The present invention will be further illustrated in the following examples.

### EXAMPLES

### Example 1

### Exemplary Omega-3 Dairy Supplement

### The following ingredients are combined to provide a feed supplement that enhances the levels of PUFA in milk derived from dairy cows fed the supplement.

### Example Formula: Omega-3 Dairy Supplement

| | | |
|---|---|---|
| Fish meal | 550 | |
| Feather meal | 138 | |
| Soybean meal | 110 | |
| Soy hulls | 42 | |
| Wheat shorts | 40 | |
| Macro min/salt | 110 | |
| Trace min/vit | 10 | |
| | 1000 | kg |

Dairy cows typically receive a daily dose of 2 kg of the supplement. Total dose may be altered for other animals.

### Example 2

### The Use of Reduced Amounts of Fishmeal and Feathermeal

Fishmeal & feather meal were fed to dairy cows at lower levels (0.9 kg & 0.225 kg/animal/day respectively) than the previously standard amounts. Unexpectedly, significant amounts of DHA were achieved in the milk.
Two herds (C, M) were fed these levels for five months. The average milk DHA levels (expressed as DHA as %/g of milk fatty acids) in 11 samples for Herd ‘C’ is 0.30% and for Herd ‘M’ is 0.33%. The results demonstrate that significant levels of DHA in milk can be achieved using reduced amounts. In view of the large dietary consumption of cows, this represents very significant cost savings.

### Example 3

### Use of Chitosan as a Protectant

Chitosan was used as a protectant in a whole herd field trial. The milk DHA level (expressed as DHA as %/g milk fatty acids) was 0.46%. These results demonstrate that chitosan is a very effective protectant that enhances the transfer of dietary PUFA to milk.

The present invention has been described with regard to one or more embodiments. However, it will be apparent to persons skilled in the art that a number of variations and modifications can be made without departing from the scope of the invention as defined in the claims.

What is claimed is:

1. A dairy product comprising at least 0.3% DHA/g of total milk fatty acids.

2. A dairy product according to claim 1 comprising at least 0.1% EPA/g of total milk fatty acids.

3. A dairy product according to claim 1 comprising at least 0.1% DPA/g of total milk fatty acids.

4. A dairy product according to claim 1 comprising at least 0.3% DHA at least 0.1% EPA, and at least 1% DPA/g of total fatty acids.

5. A dairy product according to claim 1 or 2, comprising a product selected from the group consisting of milk, yogurt, cheese, cream cheese, cream, butter, and ice cream.

6. A dairy product according to claim 5, wherein the product is milk.

7. A dairy product according to claim 5, wherein the product is yogurt.

8. A dairy product according to claim 5, wherein the product is cheese.

9. A dairy product according to claim 5, wherein the product is cream.

10. A dairy product according to claim 5, wherein the product is butter.

11. A dairy product according to claim 5, wherein the product is ice cream.

12. A dairy product according to claim 5, wherein the product is cream cheese.

13. A milk product according to claim 1 comprising at least 0.35% DHA.

14. A milk product according to claim 1 comprising at least 0.4% DHA.

15. A method of obtaining a dairy product as defined in claim 1, said method comprising feeding to a dairy cow a composition comprising fishmeal having at least 1.0 g DHA/100 g fatty acids.

16. A method of preventing or treating cardiovascular disease comprising administering a milk product having at least 0.3% DHA/g of total fatty adds.

17. A feed product for dairy cows comprising a source of PUFA containing at least 8% PUFA wt/wt and a protectant.

18. A feed product according to claim 17 comprising fishmeal as a source of PUFA.

19. A feed product according to claim 18 wherein the fishmeal comprises at least about 1 g DHA/100 g FA.

20. A feed product according to claim 19 wherein the fishmeal comprises at least 1.2 g DHA/100 g FA.

21. A feed product according to claim 17, wherein the protectant is selected from the group consisting of chitin, chitosan, feathermeal, zein, ionophore, lignin, metal salts and combinations thereof.

22. A food product comprising a dairy product according to claim 1.

23. A food product according to claim 22, wherein the food product is selected from the group consisting of soup, sauce, gravy, prepared foods and baby formula.
